(19) United States
(12) Patent Application Publication
(10) Pub. No.: US 2004/0131689 A1
(43) Pub. Date: Jul. 8, 2004

(54) Method to obtain microparticles containing a h+, k+ -atp-ase inhibitor

(76) Inventor: Brita Sjoblom, Molndal (SE)

Correspondence Address: WHITE & CASE LLP, PATENT DEPARTMENT, 1155 AVENUE OF THE AMERICAS, NEW YORK, NY, 10036, US

(21) Appl. No.: 10/469,888

(22) PCT Filed: Mar. 6, 2002
(86) PCT No.: PCT/SE02/00399

(30) Foreign Application Priority Data
Mar. 9, 2001 (SE) 0100823-4

(51) Int. Cl. A61K 9/14; B29B 9/00

(52) U.S. Cl. 424/489; 264/5

(57) ABSTRACT

A method for the preparation of homogeneous microparticles containing a H⁺,K⁺-ATPase inhibitor by a spray freezing technique characterized in that the medium to be atomized into droplets is having a high solid content and comprising besides the acid labile H⁺,K⁺-ATPase inhibitor also a polymer and a liquid in which the polymer is soluble.

### FIELD OF INVENTION

The present invention provides microparticles containing an acid labile H⁺,K⁺-ATPase inhibitor and a method of obtaining such microparticles using a spray freezing technique.

### BACKGROUND OF THE INVENTION

The strategy for the development of a pharmaceutical formulation of a given drug depends on different factors. Ultimately, these factors emanate from 1) the therapeutic needs, 2) the physical and chemical properties of the drug, and 3) the influence from the biological environment where the formulation should release its contents. Thus, both technical and biopharmaceutical considerations will contribute to a successful therapy.

However, improved drug administration can be achieved by modified release of the drug from the pharmaceutical formulation, which has been discussed extensively in the literature, e. g., R L Langer and D L Wise (Eds) “Medical Applications of Controlled Release”, vols I, II (1984), CRC Press Inc, Boca Raton.

Several approaches to achieve different types of modified release are described in the references above. Of special importance to the present invention is formulating the active substance with a suitable carrier material into microparticles. Such a formulation contains a multitude of discrete delivery units, which each can be coated, if necessary with, e.g., a suitable pH sensitive, semipermeable or other polymeric film, preferably an enteric coating. Several advantages can be obtained with this type of formulation compared to conventional tablets. Thus, the small size of the microparticles assures a fast and predictable emptying from the stomach, which is of special importance in the presence of food. Controllable plasma levels of absorbed drug can also be obtained. From a technological point of view, microparticles are more suitable for coating and handling since a technical fault during the process is fatal for single unit formulations but less so for multiple unit formulations comprising micropellets. Also, microparticle formulations are more versatile for use in different dosage strengths.

An ideal method for the preparation of microparticles where the drug is homogeneously distributed should be simple, reproducible, rapid and independent on the solubility characteristics of the drug. A high yield of the active substance in the final microparticles should also be obtained.

Several different techniques are available for making microparticles (<1 mm), e.g., spray-drying, extrusion-spheronization, spray-chilling, emulsion solvent evaporation/extraction and coating of nonpareil spheres among others. A review by Conti et al. STP Pharma. Sci. 7, 331 (1997) discusses the technical aspects of coacervation, spray-drying, emulsion solvent extraction, and emulsion solvent evaporation.

However, all existing techniques suffer from one or more drawbacks. Thus, many drugs are sensitive to heat and will deteriorate during processing.

In extrusion spheronization and in coating of non-pareil particles it has been difficult to achieve acceptable microparticles in the range of 50-400 μm. Pellets made by these methods by necessity contain significant amounts of inert excipients.

Finally, in emulsification solvent evaporation, an emulsion has to be made. This involves risking the degradation of an acid labile H⁺,K⁺-ATPase inhibitor during processing and restricts the use of this technique. Another drawback is the toxicity of the solvent used, usually methylene chloride, which can remain in the microparticles after drying.

However, despite the many different approaches there has not been disclosed a technique that can produce microparticles with high porosity in low processing temperatures. Small particles of uniform size improves segregation and dose variation during further processing into capsules or tablets. Microparticles of high porosity allow good release of the drug. Desirable aspects such as low processing temperature allows the possibility to produce spherical microparticles of different size ranges that are homogeneous, have a high drug content and sufficient mechanical strength (to e.g., withstand coating processes) into one single technique.

Spray-freezing technique has been used for the processing and granulation of ceramic materials to achieve homogeneous distribution of additives within granules to be compacted. For the processing of slurries containing silicon-nitride, sintering additives and a binder, spherical free-flowing granules were prepared by spray-freezing and subsequent freeze-drying. The homogeneity of the slurry was retained in the granules and thus in the final sintered product (Nyberg et al, Euro-Ceramics II 1, 447 (1993)). Suspensions of silicon carbide and additives were processed in this way to give granules for compaction (U.S. Pat. No. 4,526,734). The increased homogeneity compared with traditional granulation techniques resulted in better mechanical properties of a whisker-reinforced ceramic (EP 0 584 051). The process was also feasible for making homogeneous powder blends for ceramic superconductors (Japanese unexamined patent application no. 59-102433).

Normally pharmaceutical materials are lyophilised by freeze-drying in a bulk process where the solution/suspension to be frozen is placed in vials or on trays in a freeze-drier, where freezing and subsequent sublimation of the solid solvent take place. The dry product is a powder cake.

The rapid freezing provided by spray-freezing ensures that no concentration gradients exist in the resulting frozen particles and degradation of biological material is prevented. This approach has been used to get precise metering and dispensing (M. J. Akers and D. J. Schmidt, BioPharm 28, (April 1997)) where the frozen particles were in the form of large lumps, 1-9 mm. Freezing of droplets in a moving bath of Freon 12 (−20° C.), which conflicts with environmental demands, was used to obtain porous, free-flowing, spherical granules with rapid dissolution; (U.S. Pat. No. 3,932,943) as well as making homogeneous granules for tableting with precise dosing (U.S. Pat. No. 3,721,725).

A process for preparing foamed bioabsorbable polymer particles for surgical use was presented in U.S. Pat. No. 5,102,983. Here, however, the porosity was very large, and the pore size was in the range of 4-10 μm. The patent also disclosed that the solid content of the solution being sprayed was 1-20 wt %.

U.S. Pat. No. 5,019,400, discloses the use of a mixture of a biologically active material, a polymer, and a solvent which was sprayed into a non-solvent cooling medium that results in the freezing of the droplets with subsequent extraction of the solvent in the droplets during heating. The particles were finally dried in a vacuum-drier. The microparticles formed were porous, but contained only between 0.01-50% of the active substance. The solid content of the solution sprayed was 6 wt %.

GB 2 3229 124 discloses a method of forming particles containing an active agent. There is no teaching in the patent regarding the percentage weight of the active substance based on the solid content, the solid content of the solution being sprayed, the porosity of the particles formed, or the mechanical strength of the particles. Moreover, the patent discloses nothing about forming particles that contain as an active ingredient a gastric proton pump inhibitor.

U.S. Pat. No. 5,405,616 discloses a method to form droplets by forcing a suspension/solution/emulsion through calibrated jets. The droplets then fall into liquid nitrogen. Due to low shear forces the size of the pellets formed is large: 0.2-12 mm, which results in a particle that has a less safe dosability than if smaller particles could have been achieved. The smallest particles achieved were 0.8-1 mm. Further, to achieve pellets having low friability, the drying step after freeze-drying was performed by thawing the pellets before conventional vacuum drying. To achieve these low friability pellets the matrix former is restricted to materials that during thawing will form a gel. The particles obtained contain equal or less than 33 wt % of the active substance.

Particle production utilising the technique described in U.S. Pat. No. 5,405,616 intuitively is a quite slow process not suitable for large-scale industrial pharmaceutical production.

### OBJECT OF THE INVENTION

An object of the present invention is to provide a method for preparing a homogeneous microparticle which includes an acid labile H⁺,K⁺- ATPase inhibitor, or an alkaline salt. thereof, or one of its single enantiomers, or an alkaline salt thereof. The method described herein does not have the drawbacks connected to the methods discussed above, e.g., methods that rely on heat or multiple solvents for drug dissolution. Instead the method described herein puts no restrictions on the drug incorporated. Further, an object is to provide a method for preparing a microparticle with high amounts of an incorporated H⁺,K⁺- ATPase inhibitor in a high-yield process, e.g., provide microparticles that have a 80 weight % of an H⁺,K⁺- ATPase inhibitor, based on the dry weight of the microparticle. Also, the invention provides a method to prepare a homogeneous microparticle with an incorporated H⁺,K⁺- ATPase inhibitor that has low friability and sufficient mechanical strength, such that the microparticle can endure coating and compressing processes.

### BRIEF DESCRIPTION OF THE DRAWINGS

FIG. 1 is a line graph showing the weight size distribution of spray-frozen esomeprazole magnesium microparticles based on the sieve analysis.

FIG. 2 is a line graph showing the weight size distribution of spray-frozen esomeprazole magnesium microparticles based on the sieve analysis.

FIG. 3 is a line graph showing the weight size distribution of spray-frozen omeprazole microparticles based on the sieve analysis.

### DISCLOSURE OF THE INVENTION

It has been found that spherical, free-flowing, homogeneous microparticles containing H⁺,K⁺- ATPase inhibitors having low friability can be obtained by spray-freezing a suspension/solution/emulsion containing an H⁺,K⁺- ATPase inhibitor, and subsequently freeze-drying the frozen microparticles. The size distribution of the prepared microparticles is in the range from 10 to 1000 μm, e.g., in the range of 50-500 μm or 100-500 μm, and the porosity is in the range between 40-85%.

More specifically, the method of the present invention includes atomizing into droplets a liquid medium having a high dry volume content and comprising: (i) a liquid medium having an acid labile H⁺,K⁺- ATPase inhibitor, or an alkaline salt thereof, or one of its single enantiomers, or an alkaline salt thereof, (ii) a water soluble or non-water soluble polymer, wherein the polymer is at least 5% by weight based on the dry content, and (iii) a liquid in which the polymer is soluble or dispersible; freezing the formed droplets in a cold medium; and sublimating the frozen liquid\vapour from the droplets to obtain dry, homogeneous microparticles. The solid content of the liquid medium can be in the range between 15 to 60 vol %. The solid content may also be expressed as 15 to 70 weight % (corresponding to 10 to 60 vol %). The content of the H⁺,K⁺- ATPase can be from 80 to 95 weight % of the weight of the dried microparticles. The polymer can be a water soluble or non-water soluble polymer. Preferably, the polymer is a water soluble polymer. The polymer used in the present invention can act as a binder, plastizer and\or a dispersing agent, and can be any polymer known in the art, e.g., a cellulose derivative, e.g., hydroxypropyl methyl cellulose (HPMC), a polysaccharide, a natural polymer, a synthetic polymer, a surfactant and mixtures thereof. The liquid in which the polymer is soluble can be water, tertiary butyl alcohol, cyclohexane, methylene chloride, methanol, ethanol and mixtures thereof. The method includes the use of a cold medium such as liquid nitrogen, liquid argon, liquid oxygen, or a cooled solvent well below the freezing point of the liquid in the suspension. Sublimation can be performed by freeze-drying.

It was surprisingly found that the microparticles produced by the method disclosed herein, despite the high porosity of the microparticles have good mechanical strength such that they can withstand coating and compressing processes. Furthermore, the particles have a uniform size are spherical. These properties are of importance when manufacturing coated particles. Particles produced by the method described herein can thus be coated with one or more polymeric film coatings such as an enteric coating. Optionally, a separating layer can be applied before the enteric coating.

Unless otherwise defined, all technical and scientific terms used herein have the same meaning as commonly understood by one of ordinary skill in the art to which this invention belongs. In the case of conflict, the present invention, including definitions will control. All publications, patents, and other references mentioned herein are incorporated by reference.

### H⁺K⁺-ATPase Inhibitors

H⁺K⁺-ATPase inhibitors, also named as gastric proton pump inhibitors, are for instance compounds known under the generic names omeprazole, esomeprazole, lansoprazole, pantoprazole, rabeprazole and leminoprazole.

H⁺K⁺-ATPase inhibitors for use in the method described herein include compounds of the general formula I, or an alkaline salt thereof, or one of its single enantiomers, or an alkaline salt thereof.

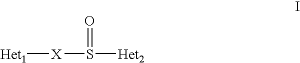

wherein

Het₁ is

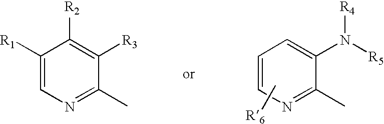

Het₂ is

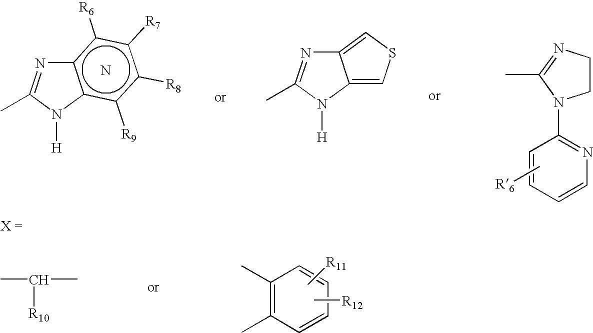

wherein

N in the benzimidazole moiety means that one of the carbon atoms substituted by R₆-R₉ optionally may be exchanged for a nitrogen atom without any substituents;

R₁, R₂ and R₃ are the same or different and selected from hydrogen, alkyl, alkoxy optionally substituted by fluorine, alkylthio, alkoxyalkoxy, dialkylamino, piperidino, morpholino, halogen, phenyl and phenylalkoxy;

R₄ and R₅ are the same or different and selected from hydrogen, alkyl and aralkyl;

R′₆ is hydrogen, halogen, trifluoromethyl, alkyl and alkoxy;

R₆-R₉ are the same or different and selected from hydrogen, alkyl, alkoxy, halogen, haloalkoxy, alkylcarbonyl, alkoxycarbonyl, oxazolyl, trifluoroalkyl, or adjacent groups R₆-R₉ form ring structures which may be further substituted;

R₁₀ is hydrogen or forms an alkylene chain together with R₃ and

R₁₁ and R₁₂ are the same or different and selected from hydrogen, halogen, alkyl or alkoxy.

The alkyl and alkoxy substituents or moieties of substituents are independently a branched or straight C₁-C₉ chain or a cyclic alkyl.

Examples of specifically interesting compounds according to formula I are:

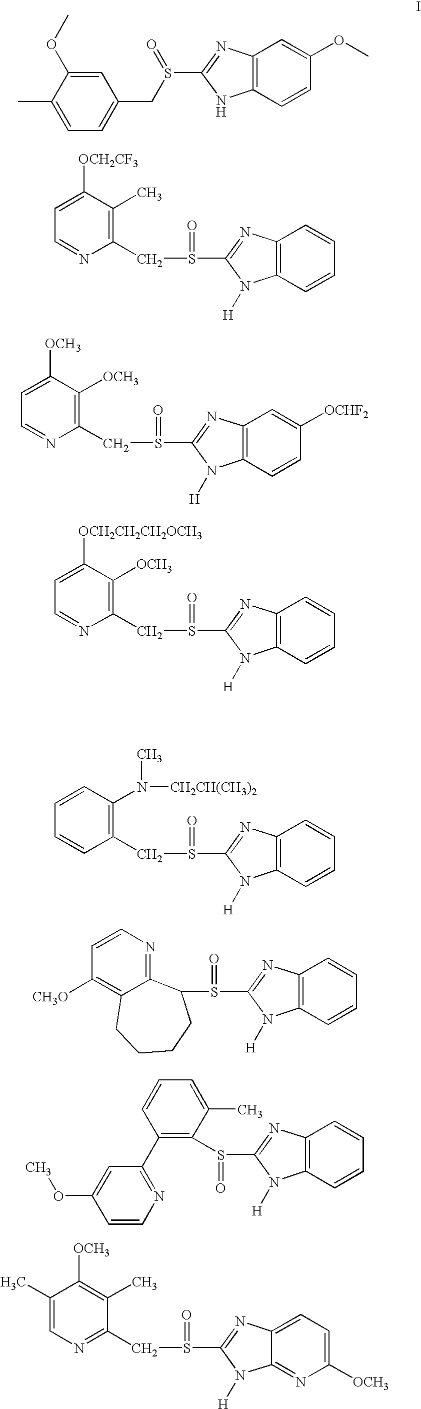

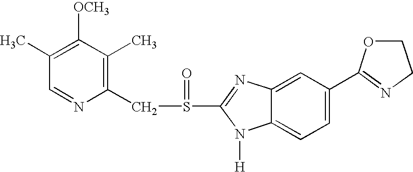

The H⁺K⁺-ATPase inhibitor used in the method of the invention may be in neutral form, or in the form of an alkaline salt, such as for instance the Mg²⁺, Ca²⁺, Na⁺ or K⁺ salts, preferably the Mg²⁺ salts. Alternatively, one of the single enantiomer or an alkaline salt thereof is used in the method of the invention.

The H⁺,K⁺-ATPase inhibitor used in the invention can be one particular H⁺,K⁺-ATPase inhibitor, e.g., omeprazole, esomeprazole magnesium, or can be a combination of different H⁺,K⁺-ATPase inhibitors.

Various different types of H⁺,K⁺-ATPase inhibitors are disclosed in EP-A1-0005129, EP-0652872, EP-0124495, EP-A1-0707580, EP-A1-174726, EP-A1-166287 and GB 2163747.

### Polymers and\or Dispersing Agents

As used herein the term polymer is intended to include any substance that can act as a binder, dispersing agent or plastizer. The polymer can be, but is not limited to, an excipient listed below:

cellulose derivatives, like ethylcellulose, hydroxypropyl methyl cellulose, hydroxyethyl cellulose, hydroxypropyl cellulose, ethyl hydroxyethyl cellulose, carboxymethyl cellulose, cellulose acetate butyrate, methylcellulose, etc

other polysaccharides, like alginate; xanthan; carrageenan; scleroglucan; pullulan; dextran; hyaluronic acid; chitin; chitosan; starch; etc

other natural polymers, like proteins (e g albumin, gelatin, etc); natural rubber; gum arabic; etc

synthetic polymers, like acrylates (e g polymethacrylate, poly(hydroxy ethyl methacrylate), poly(methyl methacrylate), poly(hydroxy ethyl methacrylate-co methyl methacrylate), Carbopol® 934, etc); polyamides (e g polyacrylamide, poly(methylen bisacrylamide), etc); polyanhydrides (e g poly(bis carboxyphenoxy)methane, etc); PEO-PPO block-co-polymers (e g poloxamers, etc); polyvinyl chloride; polyvinyl pyrrolidone; polyvinyl acetate; polyvinyl alcohol; polyethylene, polyethylene glycols and co-polymers thereof; polyethylene oxides and co-polymers thereof; polypropylene and co-polymers thereof; polystyrene; polyesters (e g poly(lactid acid), poly(glycolic acid), poly(caprolactone), etc, and co-polymers therof, and poly(ortho esters), and co-polymers thereof); polycarbonate; cellophane; silicones (e g poly (dimethylsiloxane), etc); polyurethanes; synthetic rubbers (e g styren butadiene rubber, isopropene rubber, etc); etc

surfactants, i.e., anionic, like sulphated fatty alcohols (e g sodium dodecyl sulphate), sulphated polyoxyethylated alcohols or sulphated oils, etc; cationic, like one from the group of quaternary ammonium and pyridinium cationic surfactants, etc; non-ionic, like one from the group of polysorbates (e g Tween), sorbitan esters (e g Span), polyoxyethylated linear fatty alcohols (e g Brij), polyoxyethylated castor oil (e g Cremophor), polyoxyethylated stearic acid (e g Myrj), etc; etc

other substances, like shellacs; waxes (e g carnauba wax, beeswax, glycowax, castor wax, etc); nylon; stearates (e g glycerol palmitostearate, glyceryl monostearate, glyceryl tristearate, stearyl alcohol, etc); lipids (e g glycerides, phospholipids, etc); paraffin; lignosulphonates; mono- or disaccharides (e.g. lactose, etc.); sugar alcohols (e.g. mannitol etc.); etc.

Also, combinations of these excipients are possible.

The excipients mentioned above could be made more ductile by introducing a plasticizer. The plasticizer could be but is not limited to the plasticizers mentioned below.

glycerin, polyethylene glycol, propylene glycol, triethyl citrate, diethyl phatalte, dibuthyl phtalate, dibutyl sebacate, sorbitol, triacetin, etc

Also, combinations of these plasticizers are possible.

### Low Friability Microparticles Containing Acid Labile H⁺,K⁺-ATPase Inhibitors

Generally the following conditions are valid to obtain low friability microparticles according to the method of the invention:

To obtain low friability microparticles the solid content of the suspension/solution/emulsion should be high, and can, for instance, be in the range of 10 to 70 weight %, 10 to 60 weight %, 15-70 weight % and 20 to 60 weight %. Expressed otherwise, low friability microparticles, that can for instance endure coating with a polymeric film, are achieved when the suspension/solution/emulsion is having a solid volume content equal or higher than 10 vol % and preferably higher than 15 vol %, more preferably up to 60 vol %. A high total content of the H⁺,K⁺-ATPase inhibitor can be obtained, for example, as much as 80 weight %, e.g., 85 weight %, 90 weight %, or 95 weight % (based upon solid content). The median pore size of the obtained microparticles being preferably equal or less than 1.0 μm. Solid content and solid volume content are weight % and volume %, respectively, of dry material in the suspension/solution/emulsion (dry/(dry+liquid)), wherein dry material is H⁺,K⁺-ATPase inhibitor+polymer.

According to the present invention homogeneous microparticles can be obtained wherein the solid volume content is from 10 to 60 vol %, preferably 15 to 60 vol % and giving dry microparticles with a relative density of 10 to 60 vol % and 15 to 60%, respectively (a porosity of 90 to 40 and 85 down to 40 vol %, respectively). [100% relative density is the density of the dry material; the weight of the dry material/the volume of the dry material. A suspension/solution/emulsion having 50 vol % dry material results in a relative density of 50% of the frozen and dry material].

The content of the H⁺,K⁺-ATPase inhibitor calculated on the weight of the dried microparticles are from 80 to 95 weight %, for example from 75 to 90 weight %. In one example, the H⁺,K⁺-ATPase inhibitor is in an amount equal or greater than 80%, e.g., 85 weight %.

The solid content of the liquid medium is defined as the residue after drying at 110° C. for 2 hours, divided by the total amount before drying. The solid content can be expressed either as weight percent or preferably as volume percent.

The success of obtaining low porous microparticles and thus low friable microparticles depends on the volume fraction of solids. The solid content of a suspension/solution/emulsion should thus preferably be expressed in volume fraction.

Thus, a microparticle according to the present invention comprises one (or several) H⁺,K⁺-ATPhase inhibitors, with one (or several) additional non-active substance, which are dispersed within the mnicrosphere.

### Methods of Making Microp Articles

The microparticles are obtained by spraying a homogeneous suspension/solution/emulsion of the active subtance(s) through an atomizer into a vessel with a cold medium with a temperature well below that of the freezing point of the liquid in the droplets. Frozen droplets will then form instantaneously. The structure of the suspension/solution/emulsion is retained in the droplets providing a homogeneous distribution of the substances within the droplets. The frozen liquid is then sublimated by freeze-drying of the frozen droplets where the structure of the droplets is retained due to no migration of substance during drying.

The following general steps of the procedure are further exemplified in the Experimental Section below:

a) Preparation of a medium for atomizing. The medium is a suspension, a solution or an emulsion of the H⁺,K⁺-ATPase inhibitor. A suspension is prepared by dissolving or dispersing a polymer in a liquid (as defined below), and then adding fine particles of the H⁺,K⁺-ATPase inhibitor. A further dispersing agent, e.g., a surfactant, might also be included to facilitate the dispersion of the active substance. The polymer might then act as a binder between the fine active substance particles in the microparticles and can be either a water soluble or a non-water soluble polymer.

b) Atomizing of the suspension/solution/emulsion into droplets. The suspension/solution/emulsion is fed by a nozzle that could be a pneumatic nozzle, an ultrasonic nozzle, a rotary atomizer or a pressurized nozzle. A typical size distribution of spheres produced by this process can range from 1000 μn down to 10 μn. Preferably the size distribution is in the range of between 50-500 μm.

c) Freezing of the formed droplets: The atomizer is situated above the cold medium in a cylindrical vessel. If the cold medium is a liquified gas the droplets in the spray formed by the nozzle hit the cold boiling gas before hitting the cold medium that is stirred to get a better wetting of the droplets. Instant freezing takes place and the structure of the homogeneous suspension is retained within the frozen microparticles.

d) Sublimation of the frozen liquid within the droplets: The frozen droplets are transferred from the cold medium to a freeze-drier to sublimate the frozen liquid. This step takes place without any shrinkage of the droplets or migration of excipients (e.g., polymers) and thus the structure of the suspension/solution/emulsion is retained within the dry microparticles.

The polymer or dispersing agent used for the formulation might be a solid polymer that is partly or fully soluble in the liquid. The polymer or dispersing agent used might also be a dispersion of polymer particles (e g a latex).

The liquid used for the preparation of the suspension/solution/emulsion can be a solvent for the excipients listed above and encompass, e.g., water or organic solvents with freezing point well above the freezing point of the medium used for freezing as exemplified below. Liquids, alone or a mixture of, suitable to make a suspension/solution/emulsion of the active substance, can then be, but are not limited to:

water (melting point (mp) 0° C.), tertiary butyl alcohol (mp 25.5° C.), cyclohexane (mp +6° C.), methylene chloride (mp −95.1° C.), acetone (mp −95.3° C.), methanol (mp −94° C.), ethanol (mp −117° C.), etc;

The cold medium can typically be a liquified gas, e g liquid nitrogen (boiling point −196° C.), liquid argon (boiling point −186° C.), liquid oxygen (boiling point −183° C.), or a cooled solvent well below the freezing point of the liquid in the suspension.

### Mechanical Strength of the Microparticles

The H⁺,K⁺-ATPase inhibitors are susceptible to degradation/transformation in acidic and neutral media. Therefore, an oral solid dosage form of microparticles must be protected from contact with the acidic gastric juice and the H⁺,K⁺-ATPase inhibitor must be transferred in intact form to that part of the gastrointestinal tract where pH is near neutral and where rapid absorption can occur.

The mechanical strength of the microparticles is dependent on a number of different factors including the porosity and the polymer content of the microparticles. The porosity of the microparticles is controlled in the method by the solid content of the suspension/solution/emulsion. Apart from the porosity, the brittleness of the microparticles is controlled by the amount of added polymer (binder) to the suspension/solution/emulsion. In order to obtain low friability particles the solid content of the suspension or solution or emulsion should be high.

The microparticles produced by the present method can be coated, e.g., with an enteric coating. In one embodiment, the dry microparticles are coated and are then put into capsules or incorporated into a tablet compressed by methods known to those skilled in the art. In another embodiment, the microparticles are compressed into tablets and the tablets are then coated.

It was surprisingly found that the method of the present invention results in microparticles that have a good mechanical strength. The microparticles produced by the present invention can withstand coating with a polymer coating in a fluidized bed. Moreover, it was surprisingly found that tablets containing enteric coated microparticles can be manufactured by compressing said microparticles into tablets without significantly affecting the properties of the enteric coating layer.

### Coating

Methods of coating particles are known in the art. For example, before applying enteric coating layer(s) onto the microparticle, the microparticle may optionally be covered with one or more separating layers comprising pharmaceutical excipients optionally including alkaline compounds such as for instance pH-buffering compounds. This/these separating layer(s) separate(s) the microparticle from the outer layer(s) being enteric coating layer(s).

The separating layer(s) can be applied to the core material by coating or layering procedures using suitable equipment such as in a fluidized bed apparatus using water and/or organic solvents for the coating process. The materials for separating layers are pharmaceutically acceptable compounds such as, for instance, sugar, polyethylene glycol, polyvinylpyrrolidone, polyvinyl alcohol, polyvinyl acetate, hydroxypropyl cellulose, methyl-cellulose, ethylcellulose, hydroxypropyl methyl cellulose, carboxymethylcellulose sodium and others, used alone or in mixtures. Additives such as plasticizers, colorants, pigments, fillers, anti-tacking and anti-static agents, such as for instance magnesium stearate, titanium dioxide, talc and other additives may also be included into the separating layer(s). The optionally applied separating layer(s) is not essential for the invention. However the separating layer(s) may improve the chemical stability of H⁺,K⁺-ATPase inhibitor and/or the physical properties of the novel multiple unit tableted dosage form.

One or more enteric coating layers are applied onto the microparticle using a suitable coating technique known in the art. The enteric coating layer material may be dispersed or dissolved in either water or in suitable organic solvents. As enteric coating layer polymers one or more, separately or in combination, of the following can be used; e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s).

The enteric coating layers may contain pharmaceutically acceptable plasticizers to obtain the desired mechanical properties, such as flexibility and hardness of the enteric coating layers. Such plasticizers are for instance, but not restricted to, triacetin, citric acid esters, phthalic acid esters, dibutyl sebacate, cetyl alcohol, polyethylene glycols, polysorbates or other plasticizers.

The amount of plasticizer is optimised for each enteric coating layer formula, in relation to selected enteric coating layer polymer(s), selected plasticizer(s) and the applied amount of said polymer(s), in such a way that the mechanical properties, i.e. flexibility and hardness of the enteric coating layer(s), for instance exemplified as Vickers hardness, are adjusted so that the acid resistance of the pellets covered with enteric coating layer(s) does not decrease significantly during the compression of pellets into tablets. The amount of plasticizer is usually above 10% by weight of the enteric coating layer polymer(s), preferably 15-50% and more preferably 20-50%. The amount of plasticizer is usually above 5% by weight when the microparticles are dispensed into capsules. Additives such as dispersants, colorants, pigments, polymers e.g. poly(ethylacrylat, methylmethacrylat), anti-tacking and anti-foaming agents may also be included into the enteric coating layer(s). Other compounds may be added to increase film thickness and to decrease diffusion of acidic gastric juices into the acidic susceptible material.

To protect the H⁺,K⁺-ATPase inhibitors and to obtain an acceptable acid resistance the enteric coating layer(s) constitutes a thickness of approximately at least 10 μm, preferably more than 20 μm. The maximum thickness of the applied enteric coating layer(s) is normally only limited by processing conditions.

### Over-Coating Layer

Microparticles covered with enteric coating layer(s) may further be covered with one or more over-coating layer(s). The over-coating layer(s) can be applied to the enteric coating layered pellets by coating or layering procedures in suitable equipments in a fluidised bed apparatus using water and/or organic solvents for the layering process. The materials for over-coating layers are pharmaceutically acceptable compounds such as, for instance sugar, polyethylene glycol, polyvinylpyrrolidone, polyvinyl alcohol, polyvinyl acetate, hydroxypropyl cellulose, methylcellulose, ethylcellulose, hydroxypropyl methylcellulose, carboxymethylcellulose sodium and others, used alone or in mixtures. Additives such as plasticizers, colorants, pigments, fillers, anti-tacking and anti-static agents, such as for instance magnesium stearate, titanium dioxide, talc and other additives may also be included into the over-coating layer(s). Said over-coating layer may further prevent potential agglomeration of enteric coating layered pellets, protect the enteric coating layer towards cracking during the compaction process and enhance the tableting process. The maximum thickness of the applied over-coating layer(s) is normally only limited by processing conditions.

The microparticles achieved can be coated with a polymer to achieve a time-controlled release, a site-controlled release or a pH-dependent release. Suitable polymers for coating can be, but are not limited to, the same type of polymers as listed above.

### Uses of the Microparticles Containing H⁺,K⁺-ATPase Inhibitors

The microparticles described herein can be given by different administration routes, but preferably administered orally. The microparticles can be processed into solutions, suspensions, emulsions, gels, tablets, effervescent tablets, powder in sachets, coated tablets or filled into capsules.

In a particularly preferred embodiment, the microparticles described herein are processed into a multiple unit tablet which has fast dissolving disintegrating properties in the oral cavity, or which can dissolve\disintegrate rapidly in water before being orally administered.

The microparticles described herein are useful for inhibiting gastric acid secretion in mammals and man. In a more general sense, they may be used for prevention and treatment of gastric acid related diseases in mammals and man, including e.g. reflux esophagitis, gastritis, duodenitis, gastric ulcer and duodenal ulcer. Furthermore, they may be used for treatment of other gastrointestinal disorders where gastric acid inhibitory effect is desirable e.g. in patients on NSAID therapy, in patients with Non Ulcer Dyspepsia, in patients with symptomatic gastro-esophageal reflux disease, and in patients with gastrinomas. They may also be used in patients in intensive care situations, in patients with acute upper gastrointestinal bleeding, pre-and postoperatively to prevent acid aspiration of gastric acid and to prevent and treat stress ulceration. Further, they may be useful in the treatment of psoriasis as well as in the treatment of Helicobacter infections and diseases related to these.

### WORKING EXAMPLES

The following examples illustrate different aspects of the invention without limiting the scope.

### Example 1

### Omeprazole Magnesium with Hydroxypropylmethylcellulose (HPMC) and Polysorbate 80

A suspension containing omeprazole magnesium was made according to the composition below:

| | |
|---|---|
| Omeprazole magnesium | 200 g |
| HPMC (6 cps) | 35.4 g |
| Polysorbate 80 | 4.00 g |
| Water | 360 g |

Weight percent of dry content in suspension: 39.9 weight % (32.3 vol %).

First, polysorbate 80 was mixed with the water. The HPMC (6 cps) was then added and dissolved during stirring with subsequent addition of omeprazole magnesium (prepared as in EP 97921045.7). The suspension was then deagglomerated by high-shear mixing. The deagglomerated suspension was fed through a pneumatic nozzle with a diameter of 1.0 mm at a speed of about 18 g/min. The pressure of the atomizer was 1 bar. The spray formed first hit the cold gas above a vessel filled with liquid nitrogen that was stirred to get a better wetting and instantaneous freezing of the droplets. The frozen droplets have a higher density that liquid nitrogen which make them sink to the bottom of the vessel. The frozen droplets/microparticles were then placed in a conventional freeze-drier with a shelf-temperature of −30° C. The primary drying was made at 0.25 mbar. The dry microparticles were free-flowing and spherical. According to scanning electron microscopy (SEM), the pores on the surface of the particles were smaller than 3 μm and they had a homogeneous structure.

Particles with the size of 75-1000 μm (50 g) were coated with a separating layer in a fluidized bed with a dispersion below:

| | |
|---|---|
| Hydroxypropylcellulose (LF) | 7.0 g |
| Talc | 12.0 g |
| Magnesium stearate | 1.0 g |
| Water | 140 g |

This resulted in 40% (w/w) coating on the basis of spray-frozen particles. The particles with a separating layer (50 g) were coated with enteric coating in a fluidized bed with a coating dispersion below:

| | |
|---|---|
| Eudragit ® L30D | 166.7 g |
| Triethyl Citrate | 15.0 g |
| Glyceryl | 2.5 g |
| monostearate | |
| Polysorbate 80 | 0.25 g |
| Water | 97.9 g |

The acid resistance of enteric coated particles after 2 hours in 0.1 M hydrochloride acid was 94%.

### Example 2

### Esomeprazole Magnesium with HPMC

A suspension containing esomeprazole magnesium was made according to the composition below:

| | |
|---|---|
| Esomeprazole magnesium | 200 g |
| HPMC (6 cps) | 35.3 g |
| Water | 383.9 g |

Weight percent of dry content in suspension: 38 weight % (31.5 vol %).

First, HPMC (6 cps) was added and dissolved in water during stirring with subsequent addition of esomeprazole magnesium (prepared as in EP 95926068.8). The suspension was then deagglomerated by high-shear mixing. The deagglomerated suspension was fed through a rotary nozzle with a diameter of 50 mm at a rotation speed of 5200 rpm and pumping rate of about 18 g/min. The spray formed first hit the cold gas above a vessel filled with liquid nitrogen that was stirred to get a better wetting and instantaneous freezing of the droplets. The frozen droplets have a higher density that liquid nitrogen which make them sink to the bottom of the vessel. The frozen droplets/microparticles were then placed in a conventional freeze-drier with a shelf-temperature of −30° C. The primary drying was made stepwise at 0.25 mbar. The dry microparticles were free-flowing and spherical. According to scanning electron microscopy (SEM), the pores on the surface of the particles were smaller than 2 μm and they had a homogeneous structure.

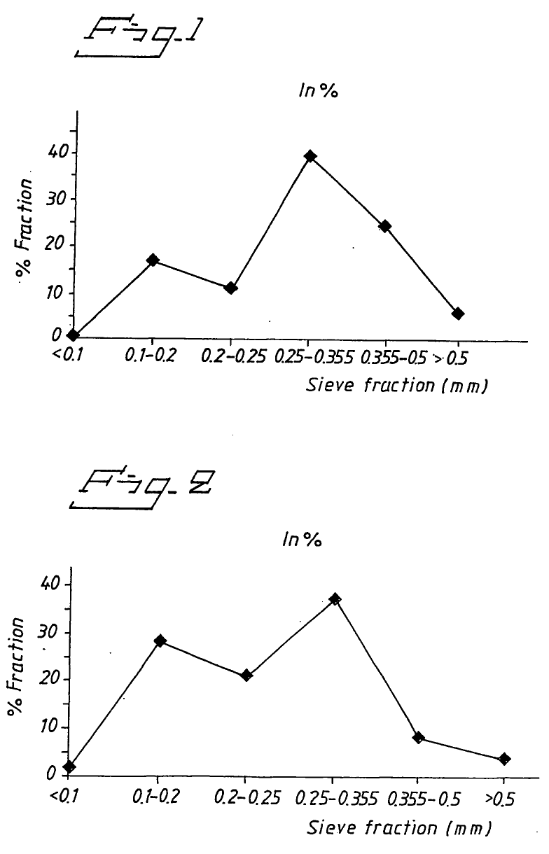

FIG. 1 shows the weight size distribution of spray-frozen esomeprazol magnesium microparticles based on the sieve analysis.

The particle size fraction of 0.20-0.25 μm (40 g) was coated with a separating layer in a fluidized bed with a dispersion below:

| | |
|---|---|
| Hydroxypropylcellulose (LF) | 45.5 g |
| Talc | 78 g |
| Magnesium stearate | 6.5 g |
| Water | 910 g |

The particle size fraction of 0.25-0.3 mm with a separating layer (70 g) was coated with enteric coating in a fluidized bed with a coating dispersion below:

| | |
|---|---|
| Eudragit ® L30D | 278.0 g |
| Triethyl citrate | 25.0 g |
| Glyceryl monostearate | 4.2 g |
| Polysorbate 80 | 0.42 g |
| Water | 163.2 g |

For the size fraction of 0.355-0.45 mm, the acid resistance of these enteric coated particles after 2 hours in 0.1 M hydrochloride acid was 94%.

The enteric coated particles were then mixed with microcrystalline cellulose for 10 min in a Turbula mixer (W.A. Bachofen, Switzerland). Sodium stearyl fumarate was then added through a sieve and the final mixture was blended for 2 min. The composition of the mixture is given below:

| | |
|---|---|
| Enteric coated particles | 40.00 w/w % |
| Microcrystalline cellulose | 59.86 w/w % |
| Sodium stearyl fumarate | 0.14 w/w % |

An amount of 194 mg of the mixture, corresponding to an esomeprazole content of 10 mg, was individually weighed for each tablet on an analytical balance and manually filled into the die of a single punch press (Korsch EK0, Germany). Compaction was then performed with 1.13 mm flat-faced punches at a maximum compaction force of 4.0±0.3 kN. The hardness of the tablets was approximately 20 N (Schleuniger, Switzerland).

The acid resistance of the tablets (comprising the enteric coated particles) after 2 h in 0.1 M hydrochloride acid was 95%.

### Example 3

### Esomeprazole Magnesium with Polyvinyl Alcohol (PVOH), Polyethylene Glycol (PEG) 400 and Polysorbate 80

A suspension containing esomeprazole magnesium was made according to the composition below:

| | |
|---|---|
| Esomeprazole magnesium | 200 g |
| Polyvinyl alcohol (10.2% solution in water) | 276.8 g |
| Polyethylene glycol 400 | 7.05 g |
| Polysorbate 80 | 4 g |
| Water | 142 g |

Weight percent of dry content in suspension: 38% (31.5 vol %).

First, polysorbate 80 was dissolved in water. Then PEG 400 was added and dissolved in water during stirring. Polyvinyl alcohol solution was added with subsequent addition of esomeprazole magnesium. The suspension was then deagglomerated by high-shear mixing. The deagglomerated suspension was fed through a rotary nozzle with a diameter of 50 mm at a rotation speed of 5200 rpm and pumping rate of about 18 g/min. The spray formed first hit the cold gas above a vessel filled with liquid nitrogen that was stirred to get a better wetting and instantaneous freezing of the droplets. The frozen droplets have a higher density that liquid nitrogen which make them sink to the bottom of the vessel. The frozen droplets/microparticles were then placed in a conventional freeze-drier with a shelf-temperature of −30° C. The primary drying was made at 0.25 mbar. The dry microparticles were free-flowing and spherical. According to scanning electron microscopy (SEM), the pores on the surface of the particles were smaller than 3 μm and they had a homogeneous structure.

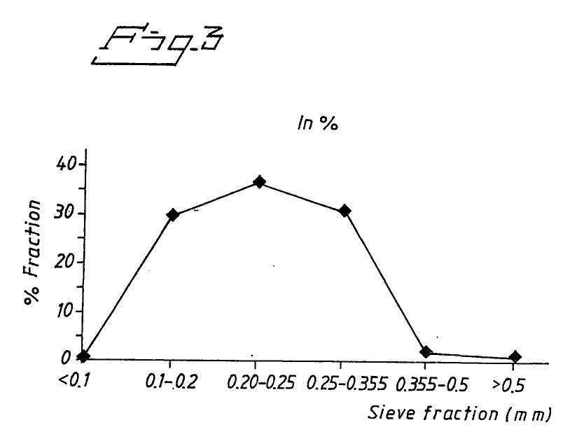

FIG. 2 shows the weight size distribution of spray-frozen esomeprazol magnesium microparticles based on the sieve analysis.

The particle size fraction of 0.20-0.25 μm (40 g) was coated with a separating layer in a fluidized bed with a dispersion below:

| | |
|---|---|
| Hydroxypropylcellulose (LF) | 40.25 g |
| Talc | 69 g |
| Magnesium stearate | 5.75 g |
| Water | 805 g |

The particle size fraction of 0.25-0.3 mm with a separating layer (40 g) was coated with enteric coating in a fluidized bed with a coating dispersion below:

| | |
|---|---|
| Eudragit ® L30D | 177.13 g |
| Triethyl citrate | 15.94 g |
| Glyceryl monostearate | 2.66 g |
| Polysorbate 80 | 0.27 g |
| Water | 104.0 g |

For the size fraction of 0.355-0.45 mm, the acid resistance of these enteric coated particles after 2 hours in 0.1 M hydrochloride acid was 93%.

The enteric coated particles were then mixed with microcrystalline cellulose for 10 min in a Turbula mixer (W.A. Bachofen, Switzerland). Sodium stearyl fumarate was then added through a sieve and the final mixture was blended for 2 min. The composition of the mixture is given below:

| | |
|---|---|
| Enteric coated particles | 36.36% |
| Microcrystalline cellulose | 63.50% |
| Sodium stearyl fumarate | 0.14% |

An amount of 174 mg of the mixture, corresponding to an esomeprazole content of 10 mg, was individually weighed for each tablet on an analytical balance and manually poured into the die of a single punch press (Korsch EK 0, Germany). Compaction was then performed with 1.13 mm flat-faced punches at a maximum compaction force of 4 kN. The hardness of the tablets was approximately 20 N (Schleuniger, Switzerland).

The acid resistance of the tablets (comprising the enteric coated particles) after 2 h in 0.1 M hydrochloride acid was 90%.

### Example 4

### Omeprazole with HPMC and Polysorbate 80

A suspension containing omeprazole was made according to the composition below;

| | |
|---|---|
| Omeprazole | 200 g |
| HPMC (6 cps) | 35.3 g |
| Polysorbate 80 | 4 g |
| Water | 390.4 g |

Weight percent of dry content in suspension: 38% (31.5 vol %).

Relative density of resulted particles (based on the dry content): 0.42 g/cm³.

First, polysorbate 80 was dissolved in water. Then HPMC was added and dissolved with subsequent addition of omeprazole. The suspension was then deagglomerated by high-shear mixing. The deagglomerated suspension was fed through a rotary nozzle with a diameter of 50 mm at a rotation speed of 5200 rpm and pumping rate of about 18 g/min. The spray formed first hit the cold gas above a vessel filled with liquid nitrogen that was stirred to get a better wetting and instantaneous freezing of the droplets. The frozen droplets have a higher density that liquid nitrogen which make them sink to the bottom of the vessel. The frozen droplets/microparticles were then placed in a conventional freeze-drier with a shelf-temperature of −30° C. The primary drying was made at 0.25 mbar. The dry microparticles were free-flowing and spherical. According to the scanning electron microscopy (SEM), the pores on the surface of the particles were smaller than 3 μm and they had a homogeneous structure.

FIG. 3 shows the weight size distribution of spray-frozen omeprazole microparticles based on the sieve analysis.

The particle size fraction of 0.20-0.25 μm (40 g) was coated with a separating layer in a fluidized bed with a dispersion below:

| | |
|---|---|
| Hydroxypropylcellulose (LF) | 75.25 g |
| Talc | 129 g |
| Magnesium stearate | 10.75 g |
| Water | 1505 g |

The particle size fraction of 0.25-0.3 mm with a separating layer (70 g) was coated with enteric coating in a fluidized bed with a coating dispersion below:

| | |
|---|---|
| Eudragit ® L30D | 201.73 g |
| Triethyl citrate | 18.15 g |
| Glyceryl monostearate | 3.03 g |
| Polysorbate 80 | 0.30 g |
| Water | 118.49 g |

For the size fraction of 0.355-0.45 mm, the acid resistance of these enteric coated particles after 2 hours in 0.1 M hydrochloride acid was 97%.

This fraction (70 g) was overcoated in a fluidized bed with a composition below:

| | |
|---|---|
| HPMC | 1.75 g |
| Magnesium stearate | 0.05 g |
| Water | 34.20 g |

For the size fraction of 0.355-0.45 mm, the acid resistance of these overcoated particles after 2 hours in 0.1 M hydrochloride acid was 98%.

The overcoated particles were mixed with microcrystalline cellulose for 10 min in a Turbula mixer (W.A. Bachofen, Switzerland). Sodium stearyl fumarate was then added through a sieve and the final mixture was blended for 2 min The composition of the mixture is given below:

| | |
|---|---|
| Enteric coated particles | 40.07% |
| Microcrystalline cellulose | 59.78% |
| Sodium stearyl fumarate | 0.14% |

An amount of 217 mg of the mixture, corresponding to an omeprazole content of 10 mg, was individually weighed for each tablet on an analytical balance and manually poured into the die of a single punch press (Korsch EK 0, Germany). Compaction was then performed with 1.13 mm flat-faced punches at a maximum compaction force of 4.0±0.3 kN. The hardness of the tablets was approximately 40 N (Schleuniger, Switzerland).

The acid resistance of the tablets (comprising the overcoated particles) after 2 h in 0.1 M hydrochloride acid was 101%.

Porosity Parameters of Spray-Frozen Microparticles (Examples 2-4) Before Coating

Total pore volume, bulk density (i.e. granular density) and median pore size were determined by mercury porosimetry (Auto Pore III (Model 9420), Micromeritics, US) by using the pressure range which corresponded to pore sizes between 0.0005 μm and 10 μm. Porosity was calculated from the bulk density and from the true density of particles measured by helium pycnometry (AccuPyc 1330, Micromeritics).

| | | | | | Total | |
|---|---|---|---|---|---|---|
| | Dry | True | Bulk | | pore | Median |
| | content | density | density | Porosity | volume | pore size |
| Example | (vol %) | (g/cm³) | (g/cm³) | (%) | (ml/g) | (μm) |
| 3 | 31.5 | 1.360 | 0.74 | 46 | 0.759 | 0.3 |
| 4 | 31.5 | 1.355 | 0.52 | 61 | 1.277 | 0.5 |

1. A method of preparing a homogeneous microparticle comprising an acid labile H⁺,K⁺-ATPase inhibitor, the method comprising:
atomizing into droplets a liquid medium having a high solid content and comprising:
(i) an acid labile H⁺,K⁺-ATPase inhibitor, an alkaline salt thereof, or one of its single enantiomers, or an alkaline salt thereof,
(ii) a polymer selected from the group consisting of a water soluble or water insoluble polymer, wherein the polymer or is at least 5% by weight based on the dry. content, and
(iii) a liquid in which the polymer is soluble or dispersible,
freezing the formed droplets in a cold medium; and
sublimating the frozen liquid\vapour from the droplets to obtain a dry, homogeneous microparticle, wherein at least 80% by weight of the microparticle based on its dry content is the weight of the acid labile H⁺,K⁺-ATPase inhibitor, the alkaline salt thereof, or one of its single enantiomers, or the alkaline salt thereof.

2. A method according to claim 1 wherein the solid content of the liquid medium is from 15 to 70 weight %.

3. A method according to claim 1 wherein the solid content of the liquid medium is from 15 to 60 weight %.

4. A method according to claim 1 wherein the liquid medium is a suspension.

5. A method according to claim 1 wherein the liquid medium is a solution.

6. A method according to claim 1 wherein the liquid medium is an emulsion.

7. A method according to any of the preceeding claims wherein the acid labile H⁺,K⁺-ATPase inhibitor has the percentage weight of 80 to 95, based on the weight of the dried microparticle.

8. A method according to any of the preceeding claims wherein the solid content of the liquid medium is from 15 to 70 percentage weight and the percentage weight of the acid labile H⁺,K⁺-ATPase inhibitor is from 80 to 95 based on the dry weight of the microparticle.

9. A method according to any of the preceeding claims wherein the polymer is selected from the group consisting of a cellulose derivative, a polysaccharide, a natural polymer, a synthetic polymer, a surfactant and mixtures thereof.

10. A method according to any of the preceeding claims wherein the liquid in which the polymer is soluble or dispersed is selected from the group consisting of water, tertiary butyl alcohol, cyclohexane, methylene chloride, methanol, ethanol and mixtures thereof.

11. A method according to any of the preceeding claims wherein the cold medium is selected from the group consisting of liquid nitrogen, liquid argon, liquid oxygen or a cooled solvent well below the freezing point of the liquid in the suspension.

12. A method according to any of the preceeding claims wherein the sublimation is performed by freeze-drying.

13. A method according to any of the preceeding claims wherein the microparticles have a size distribution in the range from 50 to 500 μm.

14. A method according to any of the preceeding claims wherein the microparticles have a size distribution in the range from 100 to 500 μm.

15. A method according to any of the preceeding claims wherein the acid labile H⁺,K⁺-ATPase inhibitor is selected from the group consisting of omeprazole, an alkaline salt thereof, esomeprazole or the alkaline salt thereof.

16. A microparticle prepared according to the method of any of claims 1-15.

17. A microparticle according to claim 16 further comprising an enteric coating.

18. A homogeneous microparticle comprising an acid labile H⁺,K⁺-ATPase inhibitor, wherein the microparticle comprises:
(i) at least 80% by weight based on the dry content of the microparticle of an acid labile H⁺,K⁺-ATPase inhibitor, or an alkaline salt thereof, or one of its single enantiomers, or an alkaline salt thereof, and
(ii) at least 5% by weight based on the dry content of a polymer, wherein the polymer is a water soluble or water insoluble polymer.

19. A microparticle according to claim 18, wherein the microparticle of claim 16 has a porosity of at least 40%.

20. A microparticle according to claim 18, wherein the microparticle has a size distribution in the range from 50 to 500 μm.

21. A microparticle according to claim 18 further comprising an enteric coating.

22. A microparticle according to claim 18 wherein the acid labile H⁺,K⁺-ATPase inhibitor is selected from the group consisting of omeprazole, an alkaline salt thereof, esomeprazole and an alkaline salt thereof.

23. A pharmaceutical composition comprising the microparticle of claim 18.

24. A method of preventing or treating a gastric acid related disease in a mammal comprising administering to the mammal an effective amount of the pharmaceutical composition of claim 23.

25. A method according to claim 24, wherein the gastric acid related disease is reflux esophagitis, gastritis, duodenitis, gastric ulcer or duodenal ulcer.

26. A use of a microparticle according to claim 18 for the preparation of a medicament for the prophylaxis or treatment of a gastric acid related disease.

27. A use of a microparticle according to claim 26, wherein the gastric acid related disease is reflux esophagitis, gastritis, duodenitis, gastric ulcer or duodenal ulcer.
